(19) United States
(12) Patent Application Publication
(10) Pub. No.: US 2014/0277583 A1
(43) Pub. Date: Sep. 18, 2014

(54) FITTING SYSTEM FOR A NEURAL ENABLED LIMB PROSTHESIS SYSTEM

(71) Applicants: SATHYAKUMAR S. KUNTAEGOWDANAHALLI, MIAMI, FL (US); RANU JUNG, MIAMI BEACH, FL (US); JAMES J. ABBAS, SCOTTSDALE, AZ (US); KENNETH HORCH, FOUNTAIN HILL, AZ (US)

(72) Inventors: SATHYAKUMAR S. KUNTAEGOWDANAHALLI, MIAMI, FL (US); RANU JUNG, MIAMI BEACH, FL (US); JAMES J. ABBAS, SCOTTSDALE, AZ (US); KENNETH HORCH, FOUNTAIN HILL, AZ (US)

(73) Assignee: THE FLORIDA INTERNATIONAL UNIVERSITY BOARD OF TRUSTEES, MIAMI, FL (US)

(21) Appl. No.: 14/216,826

(22) Filed: Mar. 17, 2014

Related U.S. Application Data
(60) Provisional application No. 61/791,679, filed on Mar. 15, 2013.

(51) Int. Cl. A61F 2/72

(52) U.S. Cl. CPC A61F 2/72
USPC 623/25

(57) ABSTRACT

Fitting systems and fitting methods for prostheses are provided. An instrumented prosthesis (IP), such as an instrumented prosthetic hand (IPH), can include sensors and can be connected to a neural stimulation system (NSS) or neural recording system (NRS), which includes an implanted system having electrodes. The NSS/NRS can also be connected to a software module, which can assist in calibrating the electrodes and mapping sensor values to stimulation parameters and/or mapping motor intent from recorded neural activity to control of prostheses.

### CROSS REFERENCE TO RELATED APPLICATION

The present application claims the benefit of U.S. Provisional Application Ser. No. 61/791,679, filed Mar. 15, 2013, which is hereby incorporated by reference herein in its entirety, including any figures, tables, and drawings.

### GOVERNMENT SUPPORT

This invention was made with government support under a grant awarded from the National Institute of Health (NIH) under grant number NIH-R0IEB008578. The government has certain rights in the invention.

### BACKGROUND OF THE INVENTION

There are more than 1.2 million individuals currently living with amputations in the United States (U.S.) alone. Of the approximately 50,000 new amputations performed each year, 25% are of the upper extremity (UE), with the remaining being lower extremity (LE). Of the UE amputations, approximately 70% occur distal to the elbow. The most common cause of UE amputation is trauma, with about three quarters of such amputations resulting from injuries in vehicle crashes, heavy machinery accidents, farm machinery accidents, industrial power tool accidents, and fire arm accidents. The incidence of UE amputation in recent conflict casualties in Iraq has also been high because of the use of body armor that leaves the limbs vulnerable.

Even though prosthetic technology continues to advance, adequate replacement of the human hand and arm remains one of the most difficult problems facing medical technology. Despite recent advancements, the current “state of the art” upper limb prosthetics remain inadequate as replacements for the lost limb. The most common causes for limb rejection and disuse include improper fit, repeated mechanical failure, high cost of repair or replacement, and dissatisfaction with overall prosthesis performance. In the U.S. alone, the overall rejection rate by upper extremity amputees is approximately 38%. When consulted about desired prosthetic limb movements and function, amputees answered differently, depending upon the type of prosthetic used (body-powered vs. myoelectric). Individuals using body-powered prostheses desired coordinated multi joint movement, increased wrist movement, and decreased requirement for visual attention, so as to be able to perform such activities as opening a door with a knob, using a spoon or fork, and fastening a button. In contrast, individuals using myoelectric prostheses desired increased digit and thumb movement and dexterity, in addition to also wanting decreased requirement for visual attention. Thus, myoelectric prosthetic users would like to be able to perform activities that involve manipulation, pointing to a strong need for sensory capabilities and finer degree of control. Many aspects of prosthetic systems can be improved, such as durability, ease of fit, and quality of fit, and many of the needs and desires of prosthetic limb users are related to the quality of sensation and control.

Body-powered and myoelectric prostheses are currently the most widely used types of upper arm prostheses. Body-powered systems translate the movement of the subject's shoulder and back muscles to control the prosthesis. The body-powered prosthesis is cheap, simple, rugged, and provides some indirect proprioceptive feedback through sense of cable position and forces transmitted by the cable. However, uncomfortable harnesses, inadequate pinch force, unnatural and exhausting body movements necessary to operate the prosthesis, and long-term pressures from the harness resulting in nerve entrapment syndromes, result in limited acceptance of the body powered prosthesis.

A myoelectric prosthesis uses electromyogram (EMG) signals from the remnant arm and/or shoulder muscles to provide control inputs to the prosthesis. In the most commonly used control scheme, myoelectric signals from an antagonist pair of muscles are used to control each degree-of-freedom (DOF) in the prosthesis, by proportionally varying the power to the actuator, as the wearer varies the strength of signal from the control muscles. For more than one DOF, the prosthesis may sequence from one actuator to the other, and simultaneous control is also available to above-elbow amputees, if independent control inputs are available from a combination of EMG and harness-mounted sensors. In the U.S., the leading manufacturers of myoelectric components include Touch Bionics, Motion Control, Liberating Technologies, and Otto Bock. As myoelectric components have improved, offering freedom from control cables (for greater comfort and range of motion), along with improved function of high pinch force, wrist rotation, and proportional control, an estimated 50% of the new arm fittings in the U.S. use myoelectric components. However, neither body-powered nor myoelectric prostheses provide true sensation from the hand for a prosthetic arm wearer.

Two approaches for providing sensory feedback have been typically considered—vibrotactile and electrotactile stimulation. However, neither of these sensory substitution systems provides input through the original sensory pathways of the amputee, and so it is not “natural”. Another, wholly different approach for providing sensory feedback is to cross anastomose severed stump nerves into intact nerve trunks allowing them to innervate intact peripheral targets (muscle or skin) One can then stimulate the cross innervated skin to provide sensory feedback. One can also record EMG signals from these cross innervated muscles in order to acquire command signals to control the prosthesis. This approach has two main limitations: the cross innervation may not be successful, leading to permanent denervation of what once was viable tissue; and functional muscle must be sacrificed to control the prosthesis. In addition, the amputee may get distally-referred sensations mixed with local sensation as the area of skin is stimulated by normal tactile input.

### BRIEF SUMMARY

Embodiments of the subject invention provide systems and methods for fitting prosthetics (e.g., neural-enabled prosthetics), for example prosthetic hands. Values from sensors of an instrumented prosthesis (IP), such as an instrumented prosthetic hand (IPH), can be mapped to stimulation parameter values on one or more electrodes implanted in or on the residual limb of the subject. The one or more electrodes can be in the vicinity of the related sensory fibers of the sensors or in the vicinity of motor fibers in the peripheral nerve. A system can include an IP having sensors, and the sensors can be, for example, for hand-opening and pinch force and/or the IP may have motors for control of the prosthesis. A system can also include a neural interface system (NIS) with a stimulation system (NSS) with an external processor to digitize and map the sensor values to stimulation parameters and/or the system can include a neural recording system (NRS) to record neural activity and map motor intent to control the prosthesis that may or may not be instrumented with sensors.

The external processor can be, for example, part of a computing device in operable communication with the remainder of the NIS, though embodiments are not limited thereto. The NIS can also include an implanted stimulator or recorder connected to electrodes implanted in the fascicles of the peripheral nerves in the residual limb (e.g., the arm). The electrodes can be, for example, longitudinal intrafascicular electrodes (LIFEs), though embodiments are not limited thereto.

Embodiments of the present invention provide fitting systems and methods for fitting a neural-enabled prosthetic system (e.g., neural-enabled prosthetic hand) that includes an IP with multiple sensors linked to an implantable stimulator system that has a multitude of electrodes interfaced with peripheral nerves. A fitting method can include: measuring electrode impedance to determine the integrity of one or more electrodes; calibrating each individual electrode to determine the stimulation current amplitude and frequency threshold and saturation values; linking individual electrodes to appropriate sensory modality; and creating and storing a map of sensory perception levels to appropriate stimulation parameters (e.g., stimulation frequency, n-let pulses for one or more instances of stimulation, jitter on interspike intervals, specification of interchannel spike timing or any other stimulation parameter known in the art) on one or more electrodes mapped to unique sensory modalities. The mapping procedure can include, for example, a perceptual modality paradigm in which the subjects use the feeling in their biological hand to determine the stimulation parameters on their amputated side.

### BRIEF DESCRIPTION OF THE DRAWINGS

FIG. 1 is a diagram of a prosthesis system that can be used with embodiments of the subject invention.

FIG. 2 is a block diagram of a system according to an embodiment of the subject invention.

FIG. 3 is a block diagram of a method according to an embodiment of the subject invention.

FIG. 4 is a diagram of a procedure that can be used according to embodiments of the subject invention.

FIG. 5 is a diagram of a procedure that can be used according to embodiments of the subject invention.

FIG. 6 is a diagram of a procedure that can be used according to embodiments of the subject invention.

FIG. 7 is a block diagram of a calibration method according to an embodiment of the subject invention.

FIG. 8 is a diagram of a system according to an embodiment of the subject invention.

FIG. 9A is an image of a sensor device according to an embodiment of the subject invention.

FIG. 9B is an image of a sensor device according to an embodiment of the subject invention.

FIG. 9C is an image of a direct input device according to an embodiment of the subject invention.

FIG. 10 is an image of a startup screen according to an embodiment of the subject invention.

FIG. 11 is an image of an impedance screen according to an embodiment of the subject invention.

FIG. 12 is an image of an impedance screen according to an embodiment of the subject invention.

FIG. 13 is an image of a calibration screen according to an embodiment of the subject invention.

FIG. 14 is an image of progress bars according to an embodiment of the subject invention.

FIG. 15 is an image of target representation in an inverse mapping method according to an embodiment of the subject invention.

FIG. 16 is a block diagram showing an experimental setup according to an embodiment of the subject invention.

### DETAILED DISCLOSURE

Embodiments of the subject invention provide systems and methods for fitting prostheses (e.g., neural-enabled prostheses), particularly prosthetic hands. Values from sensors of an instrumented prosthesis (IP), such as an instrumented prosthetic hand (IPH) that may or may not be controlled by motors, can be mapped to stimulation parameter values on one or more electrodes in or on the residual limb of the subject. The one or more electrodes can be in the vicinity of the related sensory fibers of the sensors or in the vicinity of motor fibers in the peripheral nerve. A system can include an IP having sensors, and the sensors can be, for example, for hand-opening and pinch force and/or the IP may have motors for control of the prosthesis. A system can also include a neural interface system (NIS) with a neural stimulation system (NSS) with an external processor to digitize and map the sensor values to stimulation parameters and/or the system can include a neural recording system (NRS) to record neural activity and map motor intent to control the prosthesis that may or may not be instrumented with sensors.

The external processor can be, for example, part of a computing device in operable communication with the remainder of the NIS, though embodiments are not limited thereto. The NISS can also include a stimulator or recorder connected to electrodes implanted in the fascicles of the peripheral nerves in the residual limb (e.g., the arm). The stimulator or recorder can be an implanted stimulator or recorder, though embodiments are not limited thereto. The electrodes can be, for example, longitudinal intrafascicular electrodes (LIFEs), though embodiments are not limited thereto.

Despite partial atrophy and degeneration, both central and peripheral motor and somatosensory pathways retain significant function for many years following amputation, potentially allowing the use of neuroprosthetic technologies to establish efferent neural control of a prosthetic limb with direct afferent neural sensory feedback. Hence, the limitations of the approaches listed in the Background section can be overcome by using a direct neural interface with the peripheral nerves of the forearm to communicate with the prosthetic hand. A key requirement for this approach to be successful is the availability of the appropriate electrode interface. Multiple electrode technologies can be used to establish a neural interface. Choice of the electrode depends on many factors, including biocompatibility, long-term stability, implantation difficulty, mechanical characteristics, electrochemical characteristics, and economics. Peripheral nerve interfaces can generally be grouped as extraneural or intraneural electrodes.

Extraneural electrodes are primarily the “cuff” type. Cuff dimensions, mechanical properties and materials, and the configuration of the active and indifferent sites are important. The main limitation of “cuff' electrodes is that their sensitivity level for both stimulation and recording lies at the level of the whole nerve. This can be problematic, as each peripheral nerve is made up of many heterogeneous motor and sensory axons, and true integration of neuroprosthetics will likely require sensitivity at the fascicular or axonal level.

To access the fascicles, an intraneural electrode approach can be used. Intraneural electrodes include regeneration electrodes (or sieve chips), multielectrode arrays, and single needle. In regeneration electrodes (or sieve chips), only a limited number of channels with nerve fibers growing through them may produce definitive recordings. The most probable issues lie in the distance of the recording site to a node or blockage of nerve conduction as fibers pass through the small holes in the array. Anatomical and physiological constraints dictate that an effective peripheral nerve interface for providing discrete, distally-referred sensations and for obtaining movement specific command signals from peripheral nerve stumps must place electrode sites within the perineurium of individual nerve fascicles. This could be accomplished using punctate penetrating arrays, or a slowly penetrating electrode, or individual intrafascicular electrodes. Although high-density multi-micro electrode arrays can allow access to a large population of fibers in the nerve and could allow recording from multiple electrodes of an array, the lead wires for multiple electrodes can produce tethering forces on the array, hence resulting in damage to the nerve. The implantation procedure also does not allow for the specific alignment of electrodes with specific fascicles, and the implanted array has to be securely fixed to the nerve.

In one embodiment, a fitting system uses LIFEs as electrodes. LIFEs t have been shown to provide largely stable neural recordings and localized neural stimulation in chronic animal studies, and can do the same in human amputee nerve stumps on a semi-chronic basis. These electrodes allow access to individual fascicles and recording site exposure of 1 mm or more, thereby providing improved recording capability. The somatotopic organization of peripheral nerves at both the fascicular and sub-fascicular levels allows directed access using LIFEs.

FIG. 1 shows a schematic diagram of a prosthesis according to an embodiment of the subject invention. That is, systems and methods of the subject invention can include elements of the prosthesis and/or can be used to fit and/or configure the prosthesis. Referring to FIG. 1, the prosthesis system has a direct neural link to peripheral nerves in an arm. In one embodiment, a system has an instrumented prosthetic hand (IPH) and an NSS. The IPH can include sensors across the hand to sense multiple modalities, such as hand opening, touch, pinch force, temperature, etc. The sensors can be present on the front of the hand, the back of the hand, or both. The NSS can include an external processor in operable communication with a fully implanted stimulator (implanted into the residual limb) through a communication system. The communication system between the external processor and the implanted stimulator can be wireless, for example, using a radio frequency (RF) link, though embodiments are not limited thereto. The stimulator can be connected via wires. Analog sensor inputs from the IPH can be digitized and mapped to appropriate stimulation parameters by the external processor. The mapped parameters can then be transferred to the implanted system through the communication system (e.g., RF link). Based on the transmitted pulse parameters, corresponding electrical impulses can be delivered to the peripheral nerve through a peripheral nerve interface attached to the implanted system. The peripheral nerve interface can be any suitable nerve interface known in the art, for example, a plurality of LIFEs.

An NSS for a limb prosthesis generally includes implanted stimulators with multiple leads containing one or more electrode contacts, though different electrode technologies may be used. Multiple electrodes can be used to map sensations representing a wide area of the phantom hand and also to gain access to different types of sensory fibers. In such a complex system, the output of the sensors from an IP can be mapped to one or more electrodes. Additionally, even on a single electrode, the sensor output can be mapped to a variety of stimulation parameters such as stimulation current level, stimulation pulse rate (stimulation frequency), stimulation pulse width, n-let pulses for one or more instances of stimulation, jitter on interspike intervals, specification of interchannel spike timing and other. Each of these parameters can be altered to provide varied sensations. Since each subject has a unique neural anatomy, an efficient and effective fitting method can be advantageous, allowing for quick and accurate mapping of the sensor values to stimulation parameters on appropriate electrodes.

Embodiments of the present invention provide fitting systems and methods for fitting a neural-enabled prosthetic limb system that includes an IP with multiple sensors linked to an implantable stimulator system that has a multitude of electrodes interfaced with peripheral nerves. A fitting method can include: measuring electrode impedance to determine the integrity of one or more electrodes; calibrating each individual electrode to determine the stimulation current amplitude and frequency threshold and saturation values; linking individual electrodes to appropriate sensory modality; and creating and storing a map of sensory perception levels to appropriate stimulation parameters (e.g., stimulation frequency or any other stimulation parameter known in the art) on one or more electrodes mapped to unique sensory modalities. The mapping procedure can include, for example, a perceptual modality paradigm in which the subjects use the feeling in their biological limb to determine the stimulation parameters on their amputated side. In further embodiments, a fitting method can include performing any function discussed herein, even if that function is only discussed in the context of a fitting system or device.

In many embodiments, a fitting system includes a software component, such as a software module with a user interface (UI) that can be operated by a user. The UI can be, e.g., a graphical user interface (GUI). In one embodiment the UI can include sections to: measure electrode impedances and determine electrode integrity; calibrate individual electrodes or a group of electrodes and link electrodes to particular sensory modality; map perception levels to stimulation parameters (e.g., stimulation frequency, n-let pulses for one or more instances of stimulation, jitter on interspike intervals, specification of interchannel spike timing, and/or any other stimulation parameter known in the art) on one or more electrodes; and/or configure the neural-enabled prosthetic hand system. In further embodiments, a fitting system can include any element discussed herein, even if such element is discussed only in the context of being part of a fitting method.

Embodiments of the subject invention provide systems, methods, and devices, which can include a software component, to fit a prosthesis (e.g., a prosthetic hand) with sensors to one or a plurality of electrodes implanted close to the sensory fibers in the peripheral nerves. The devices can include one or more elements to link implanted electrodes to different sensory modalities based on elicited perception on stimulation of sensory nerves. A system can include one or more external sensor devices to monitor movements and operation on both sides of a subject's body.

Embodiments of the subject invention provide systems, methods, and devices that determine appropriate stimulation levels to achieve graded perception during stimulation of sensory nerves. A system can include a perception modality matching method to map information from different types of sensors on a prosthetic hand to stimulation parameters of corresponding electrodes (e.g., electrodes linked to a particular sensory modality).

Embodiments of the subject invention provide systems, methods, and devices that include a perception modality matching method to map information from different types of sensors on a prosthetic hand to stimulation parameters of corresponding electrodes (e.g., electrodes linked to a particular sensory modality). For example, a method can include: having a subject match the perception of a given modality that is elicited by electrical stimulation on one side of the body (e.g., arm/hand) with the same modality on the other side (e.g., arm/hand); or an inverse paradigm in which a subject positions his or her intact side (e.g., arm/hand) to match a target posture and then uses a direct input device to adjust stimulation on the other side of the body (e.g., arm/hand) to match the perception on the intact hand.

Embodiments of the subject invention provide systems, methods, and devices that include a software component having a software module with a UI system suitable for providing a user with a method for fitting a hand prosthesis system having one or more implanted electrodes. For example, the UI system can include: visual representation of electrode placement in the nerve; means of selecting one or more electrodes for calibration, wherein selected electrodes for stimulation provide a perception of sensation of a particular modality; and means of mapping information from one type of sensor to stimulation frequency on one or a plurality of electrodes linked to a corresponding sensory modality. The software can include a means of selecting one or more electrodes to check electrode integrity by estimating electrode impedance. For each electrode, the second screen can include options (controls) to select a specific calibration routine and adjust the routine configuration parameters appropriately. The second screen can include a graphical display of the stimulation parameters presented, and the second screen can include a means by which the user can graphically mark the location and modality of sensation. Different levels of sensor values can be mapped to different stimulation frequency values. The third screen can include options to generate a list of target values for both a forward paradigm and inverse paradigm discussed herein. The screen can also include an option of retrieving a previously generated target list stored on the computer (or any other data storage unit). The third screen can include options to provide the experimenter and the subject with a visual feedback of data obtained from the sensors connected to the subject. The third screen can include options to provide the experimenter with options to configure the sensors connected to the computer. As part of the inverse paradigm discussed herein, the fitting system can include a means by which the target level can be presented to the subject without any indication of the absolute value. The visual feedback system can provide information about relative distance from the target.

Embodiments of the subject invention provide systems, methods, and devices that can fit a limb extremity (e.g., hand, arm or portion thereof, foot, leg or portion thereof) prosthesis with sensors with one or more electrodes implanted close to the sensory fibers in the peripheral nerves. A limb extremity prosthesis can be fitted with sensors with one more electrodes implanted in spinal or brain neural tissue that is responsive to sensory input from the limbs. In one embodiment, an external device can be fitted, such as a telerobotic system that provides sensory feedback to the operator with one or more electrodes implanted close to the sensory fibers in the peripheral nerves. In a further embodiment, an external device can be fitted, such as a telerobotic system that provides sensory feedback to the operator with one or more electrodes implanted in spinal or brain neural tissue that is responsive to sensory input from the limbs.

FIG. 2 is a block diagram for a system for fitting a prosthesis, according to an embodiment of the subject invention. Referring to FIG. 2, a system can include an IPH in operable communication with an NSS, which is in operable communication with peripheral nerves and with a software component (e.g., custom software). Each component can be in operable communication with other components, for example, through wires or wirelessly.

The software component can include a software module stored on one or more computer-readable media (e.g., non-transitory media). The software component can be stored on, for example, a compact disc (CD), digital video disc (DVD), flash memory device, volatile memory, or a hard disk drive (HDD), such as an external HDD or the HDD of a computing device.

In many embodiments, the software component is used via a computing device, and the software is either stored on a portion of the computing device (e.g., HDD or volatile memory) or is on a computer-readable medium in operable communication with and/or being read by the computing device. A computing device can be, for example, a laptop computer, desktop computer, server, cell phone, or tablet, though embodiments are not limited thereto. A user can operate the software component through a UI (e.g., a GUI) of the software component, which allows the user to interact with the software via the computing device. In one embodiment, the software component is stored on a laptop computer, tablet, or desktop computer.

FIG. 3 is a block diagram of a method of fitting a prosthesis according to an embodiment of the subject invention. Referring to FIG. 3, electrode impedances can be measured to determine the integrity of all implanted electrodes. All viable electrodes can then be calibrated to determine the limits of different stimulation parameters. During the calibration process, the electrodes can also be linked to a particular sensory modality. The mapping between the different sensory perception values and the stimulation parameters can be determined. In one embodiment, the sensory perception values are mapped to different stimulation frequency values.

In one embodiment, limits of stimulation amplitude and frequency are determined, for example for a biphasic pulse (cathodic first) with a constant pulse width and inter-phase gap value. These limits can be determined as the electrodes are calibrated (e.g., as part of the calibration process) or after the electrodes are calibrated.

During the calibration step, the goal is to determine the limiting values of different stimulation parameters. The calibration step can include determining amplitude threshold, amplitude saturation, stimulation amplitude, frequency threshold, frequency high, and/or frequency saturation. These parameters can be determined using the software component (e.g., through a GUI on a computing device), in concert with the electrodes and input from the subject. The values of any or all of these parameters can remain the same or can change from subject to subject.

The amplitude threshold is the minimum stimulation current level which elicits any sensation from the subject. In one embodiment, amplitude threshold is determined using the staircase procedure shown in the flowchart in FIG. 4. The subject can be presented with a series of pulses, and the stimulation amplitude threshold can be determined using the staircase method of FIG. 4.

For the determination of the amplitude threshold, the train of pulses can have a duration of, for example, 1 millisecond (ms) to 3000 ms and can have a frequency of, for example, 1 Hertz (Hz) to 1000 Hz. The pulses can be, for example, biphasic pulses (e.g., cathodic-first biphasic pulses). The pulse width can be fixed at a value of, for example, 50 microsecond (μs) to 1000 μs, and the inter-phase gap can be fixed at a value of, for example, 1 is to 100 μs. In one embodiment, the subject is presented with a series of 300 ms to 1000 ms train of pulses of 50 Hz to 100 Hz. The pulses are biphasic pulses (cathodic first). The pulse width is fixed at a value of 200 μs to 400 μs, and the inter-phase gap is fixed at a value of 20 μs to 40 μs.

The amplitude saturation is the maximum stimulation current level that elicits a comfortable sensation from the subject at a fixed frequency. Amplitude saturation can be determined using the method of limits procedure shown in the flowchart in FIG. 5. The subject can be presented with a series of pulses, and the stimulation amplitude saturation can be determined using the method of limits of FIG. 5.

For the determination of the amplitude saturation, the train of pulses can have a duration of, for example, 1 ms to 3000 ms and can have a frequency of, for example, 1 Hz to 1000 Hz. The pulses can be, for example, biphasic pulses (e.g., cathodic-first biphasic pulses). The pulse width can be fixed at a value of, for example, 50 μs to 1000 μs, and the inter-phase gap can be fixed at a value of, for example, 1 μs to 100 μs. In one embodiment, the subject is presented with a series of 300 ms to 1000 ms train of pulses of 50 Hz to 100 Hz. The pulses are biphasic pulses (cathodic first). The pulse width is fixed at a value of 200 μs to 400 μs, and the inter-phase gap is fixed at a value of 20 μs to 40 μs.

In one embodiment, the stimulation current amplitude is the average of the amplitude threshold value and the amplitude saturation value. In an alternative embodiment, the stimulation amplitude value is chosen as a percentage of the total range of amplitude values (threshold-saturation).

The frequency threshold is the lowest stimulation frequency which provides a fused sensation (e.g., proprioception or pinch force) in the subject, instead of a pulsating one. The stimulus current amplitude can be fixed at the previously-determined value. Similar to the amplitude threshold, the frequency threshold value can be determined using the staircase method shown in FIG. 4. The procedure is similar to the one described in the flowchart in FIG. 4, except that in this case the subject is asked if the stimulus is fused (not pulsating).

For the determination of the frequency threshold, the train of pulses can have a duration of, for example, 1 ms to 3000 ms and can have a frequency of, for example, 1 Hz to 1000 Hz. The pulses can be, for example, biphasic pulses (e.g., cathodic-first biphasic pulses). The pulse width can be fixed at a value of, for example, 50 μs to 1000 μs, and the inter-phase gap can be fixed at a value of, for example, 1 μs to 100 μs. In one embodiment, the subject is presented with a series of 300 ms to 1000 ms train of pulses of 10 Hz to 100 Hz. The pulses are biphasic pulses (cathodic first). The pulse width is fixed at a value of 200 μs to 400 μs, and the inter-phase gap is fixed at a value of 20 μs to 40 μs.

The frequency high is the highest frequency value that provides the subject with a sensation (e.g., proprioception or pinch force) that is qualitatively similar to the sensation elicited by frequency threshold value. This value can be determined using the method of limits shown in the flowchart in FIG. 5. However, after each successive increasing burst the subject is asked to name the type of sensation (e.g., proprioception or pinch force), and the frequency high is determined when the sensation changes or the maximum safe limit is reached. The maximum safe limit can be, for example, chosen by a user of the system and can be entered in through the software component.

For the determination of the frequency high, the train of pulses can have a duration of, for example, 1 ms to 3000 ms and can have a frequency of, for example, 1 Hz to 1000 Hz. The pulses can be, for example, biphasic pulses (e.g., cathodic-first biphasic pulses). The pulse width can be fixed at a value of, for example, 50 μs to 1000 μs, and the inter-phase gap can be fixed at a value of, for example, 1 μs to 100 μs. In one embodiment, the subject is presented with a series of 300 ms to 1000 ms train of pulses of 10 Hz to 400 Hz. The pulses are biphasic pulses (cathodic first). The pulse width is fixed at a value of 200 μs to 400 μs, and the inter-phase gap is fixed at a value of 20 μs to 40 μs.

The frequency saturation is the highest frequency of stimulation that provides a sensation to the subject that is indistinguishable from that perceived at frequency high. This value can be determined using the just noticeable difference procedure shown in the flowchart in FIG. 6. The stimulation parameter values for each step of the calibration procedure can be altered to suit the needs of a particular subject. This can be done by, for example, a user of the system, such as a clinician, doctor, or other medical professional.

In addition to amplitude threshold, amplitude saturation, stimulation amplitude, frequency threshold, frequency high, and frequency saturation, the electrode can be linked to a particular sensory modality during the calibration routine. FIG. 7 is a block diagram of a calibration procedure according to an embodiment of the subject invention.

Values from sensors on an instrumented prosthesis (e.g., an IPH) can be mapped to appropriate electrodes. For example, the output of the hand-opening sensor can be mapped to electrodes that provide proprioceptive perception. Similarly, the output of the pinch force on the thumb can be mapped to electrodes that provide perception of pinch force when stimulated. In many embodiments, mapping of the sensor values is performed after calibrating the electrodes.

By modulating the stimulation frequency delivered to LIFEs implanted in the fascicles of median and ulnar nerves, the magnitude of sensation (not the modality or topography) of proprioception or pinch force can be varied. Hence, in one embodiment, a method is provided for mapping hand opening and pinch force sensor values from an IPH to a range of stimulation frequencies on the appropriate electrodes.

According to certain embodiments of the present invention, a fitting method that provides mapping includes an experimental paradigm that uses ‘perceptual modality matching’. In this paradigm, the subject must match the perception of a given modality that is elicited by electrical stimulation on one site on the body with the same or different modality on another site. For example, when the electrical stimulation in the residual limb elicits a perception of hand opening (e.g., in response to stimulus train), a subject must open the intact hand to a degree that matches the perception. FIG. 8 shows a schematic of a mapping method according to an embodiment of the subject invention. Referring to FIG. 8, sensors placed on the intact hand can measure hand opening. When the electrical stimulation in the residual limb elicits a perception of grasp force, a subject must grasp an object (e.g., force sensor) with the intact hand to a degree that matches the perception.

In one embodiment, a fitting method provides an inverse paradigm in which a subject first positions his or her intact hand in a target posture or grasp an instrumented object (e.g., force sensor) with a target force level, and then adjusts the electrical stimulation level on the residual limb to match the perception on the intact side. A subject can use a direct input device to adjust stimulation pulse frequency within the range of values that was determined to be suitable for that electrode during the calibration process as shown in FIG. 8. Such a direct input device can be, for example, a foot pedal. Data collected from this process can provide quantitative measures of the relationship between stimulation level and perception. While systems and methods described relate to a hand prosthesis system with only two types of sensors, it is to be understood that the same principles can be extended to other sensory modalities including vibration, pressure, touch, and temperature.

FIGS. 9A, 9B, and 9C show sensors and equipment that can be used with systems and methods according to certain embodiments of the subject invention. Referring to FIG. 9A, a custom hand opening sensor can measure hand opening of a subject's intact hand. Referring to FIG. 9B, a custom pinch force sensor can measure the level of pinch force for a subject. Referring to FIG. 9C a custom-instrumented foot pedal can be used as a direct input device to allow a subject direct control over the level of stimulation.

In many embodiments, a fitting system includes a software component including a custom software module with a GUI. The custom software module can include multiple modules to perform functions of the fitting process. Modules that can be included in the software module, include, but are not limited to, a startup module, an impedance module, a calibration module, a fitting module, a configuration module, and a safety module.

In one embodiment, the software module includes a startup module, an impedance module, a calibration module, a fitting module, and a configuration module. The startup Module can be configured to handle subject and experimental session information, and the impedance module can be configured to execute electrode integrity checks. The calibration module can be configured to calibrate electrodes individually or in a groups, and the fitting module can be configured to map information from sensors in the prosthesis to corresponding stimulation parameters on appropriate electrodes. The configuration module can be configured to configure the NSS with electrode calibration and map parameters. In a further embodiment, the software module includes a safety module having checks to ensure that all stimulation parameters passed onto the implanted system during the fitting process are within safety limits. The software module can also include checks to make sure that the NSS is configured correctly by reading back the information written into the processor.

The startup module can be designed to handle subject and experimental session information. The startup module can provide the experimenter (i.e., the user) with options to create a new subject database, edit existing subject information, or choose a subject to start experimental session. It can also include options to allow the experimenter to load previous session information or archive current session information. FIG. 10 is an image of a startup screen of a startup module, as seen in a GUI of a computing device running the software component according to an embodiment of the subject invention. Referring to FIG. 10, a top panel can contain widgets to interact with the NSS. The top panel can also include widgets to display subject information. The startup screen shown in FIG. 10 also shows a selectable list of different subject databases (subject list box). The experimenter can choose a subject to start the experimental/study session with.

Once the study session is initiated with a given subject, the next step can be to determine the integrity of the implanted electrodes. The electrode integrity can be evaluated by measuring electrode impedances. In one embodiment, the impedance screen of the impedance module, as displayed by a GUI on a computing device, includes options to measure impedance of one or more implanted electrodes. In a further embodiment, a graphical representation of electrodes in a fascicle is included. FIG. 11 shows such a graphical representation of electrodes in a fascicle, as displayed by a GUI. Referring to FIG. 11, the software can allow the user to choose the electrode by clicking on each individual electrode or drawing a box around multiple electrodes. The measured impedance values can appear directly below the electrode or as a list on the side of the nerve cross-section picture. A coloring scheme can be employed to easily identify the broken and viable electrodes. In an alternative embodiment, the impedance module, as displayed on a GUI, includes a simple screen. FIG. 12 shows an image of such a simple screen of an impedance module as displayed in a GUI. Referring to FIG. 12, the simple screen of an impedance module can have a list of electrode numbers and corresponding impedance values. Depending on the measured impedance value, the software may classify the electrodes as “short” or “open” or “Ok” electrodes.

FIG. 13 is an image of a screen of the calibration module displayed in a GUI, according to one embodiment of the subject invention. Referring to FIG. 13, the screen of the calibration module, as displayed in a GUI, can be divided into six parts, a subject entry box, a parameter box, a test mode box, a pulse timing box, an electrode information box, and a display box. Sections of the subject entry box are described in Table 1, and sections of the parameter box, the test mode box, the pulse timing box, and the electrode information box are described in Table 2. The letters corresponding to the parameter box (A), the test mode box (B), the pulse timing box (C), the electrode information box (D), and the display box (E) are indicated on FIG. 13. Also, the subject entry boxy is labeled as such on FIG. 13.

The display box includes a plot that provides a graphical representation of the stimulation values being presented to the subject during the calibration routine. In one embodiment, the calibration module includes a graphical representation of the hand (a separate image of both front and back sides). The software can allow the experimenter to choose a particular region in the hand and link it to a particular electrode for a given modality.

In many embodiments, the calibration module can include a settings file, which can include settings for various parameters, as discussed in Table 2.

TABLE 1
Description of Subject Entry Box of Screen in Calibration Mode

| Widget Name | Description |
|---|---|
| Select | Select one of the calibration routines |
| Parameter | |
| Start | Start/stop calibration routine |
| Subject | Displays the question seen by the subject. Experimenter |
| response entry | can enter subject's response by clicking the appropriate |
| box | button |

TABLE 2
Description of Boxes of Screen in Calibration Mode

| | |
|---|---|
| Widget Name | Description |

A - Parameter Box: This box contains widgets pertaining to parameter chosen. The chosen
parameter is shown as the title of this box.

| | |
|---|---|
| Test Mode | Choice box allowing the user to select the procedure to estimate the |
| | chosen parameter. |
| Min | Minimum amplitude (μA) or frequency (Hz) for the procedure. The |
| | range of values accepted by this widget is set in the settings file. |
| Max | Maximum amplitude (μA) or frequency (Hz) for the procedure. The |
| | range of values accepted by this widget is set in the settings file. |
| Starting Amp | Starting value for the procedure. The range of values accepted by this |
| | widget is set in the settings file. |
| Stim Freq (Hz) | Only relevant for amplitude routines. Defines the stimulation frequency |
| | for estimating amplitude threshold and amplitude saturation. Default |
| | value is 100 Hz. |

B - Test Mode Box: This box contains widgets to configure the procedure. The chosen test
mode is shown as the title of this box.

| | |
|---|---|
| MOL Step (%) | Percentage step size for method of limits. |
| | New value = Old Value + (% step size/100) * (Old Value) |
| Step Direction | Direction of change from starting value (Increase/Decrease) |
| Coarse Step (%) | Coarse step for staircase (percent value) |
| Fine Step (%) | Fine step for staircase (percent value) |
| Number of | Number of reversals expected for staircase procedure |
| Reversals | |

C - Pulse Timing: This box contains widgets to configure the burst timings as described in
the Appendix section. All times displayed in the section are in msec. The range for all three
widgets is 1-999 msec.

| | |
|---|---|
| Burst-ON | The duration for which the stimulation burst is given. Range: 1-999 msec |
| Display Response | The duration for which the subject's response is displayed to them after |
| | the user enters it |
| Wait Time | The delay between when the time when the subject sees his response and |
| | when the subsequent stimulation is provided |

D - Electrode Information Box: This box contains widgets to list the parameters
determined during calibration. It also has widgets to allow the user to enter additional
information about the electrode.

| | |
|---|---|
| Electrode Number | Display the number of electrode being calibrated |
| Previous | Move to previous electrode |
| Next | Move to next electrode |
| Update and Save | Store calibration parameters. Generates new configuration file. |
| Stim Amp (μA) | Average of Amplitude Threshold and Amplitude Saturation |
| Modality Code | Position (proprioception) or Force or Other |
| Functional State | Same as in Impedance panel |
| Electrode Code | Assign code to electrodes |
| Phase width | This parameter should be set before calibration is initiated. Phase width in |
| | μs. The range of values accepted by this widget is set in the settings file. |
| Inter-phase gap | This parameter should be set before calibration is initiated. Phase width in |
| | μs. The range of values accepted by this widget is set in the settings file. |
| Modality | Short description of what the subjects say they are feeling |
| Description | |

Once the electrodes are calibrated and grouped under an appropriate sensory modality (e.g., pinch force or proprioception), the next step is to map the sensor values to different stimulation pulse frequency values on one or more electrodes. In one embodiment, the mapping process is carried out using the “perception modality matching” paradigm, which is a two-step process described herein. The GUI for each step will now be described.

During the forward mapping experimental paradigm, a subject can be electrically stimulated at different pulse frequency levels through one or more electrodes on the amputated side and can be instructed to indicate the perceived sensation using his or her intact arm. Sensors on the intact arm can be used to monitor the subject's response. The GUI for the fitting module can include options to allow the user to: choose one or more electrodes linked to a particular sensory modality; and/or choose a text file with a list of stimulation frequency values to be tested.

The text file can contain, for example an absolute value of stimulation frequency or percentage values. In the latter case, the stimulus provided to the subject is a percentage of the permitted frequency range (Ffused-Fsaturation). The percentage values are preferred to keep the text file independent of the electrode frequency range. The software can also include display options to allow the user to monitor the output of the sensors on the subject's hand. In one embodiment, the information about the sensor is provided through a graphical hand that opens and closes based on the input from the hand-opening sensor on the subject's intact hand. The output of the pinch force sensor on the intact hand can be mapped to a range of colors (e.g., minimum indicated by green to maximum indicated by red) shown at the tip of the thumb in the hand graphic. In an alternative embodiment, the output of the sensors is shown using progress bars. FIG. 14 is an image showing an example of such progress bars. In both cases, the sensor output may or may not be shown to the subject on a different window on a separate screen. During the course of the routine, the subject can be presented with multiple stimulus frequency values with the full range and the corresponding perception levels indicated by the subject can be stored. At the end of the routine, the software module can include an option to plot the perception levels as a function of pulse frequency. The software module can automatically show the plot at the end of the routine, or it can have an option where the experimenter can manually choose to view and save the plot.

As part of the inverse mapping experimental paradigm, a subject can first position his or her intact hand in a target posture or grasp an instrumented object (e.g., a force sensor) with a target force level, and then the subject can adjust the electrical stimulation level on the residual limb to match the perception on the intact side using a direct input device (e.g., a foot pedal). In one embodiment the GUI for the fitting module includes an option to allow the user to choose a text file with a list of target values. The text file can contain absolute target values or percentage (of full range) target values. As with the previous case, the percentage target values are preferred to keep the text file independent of the sensor device or the amputee.

For this paradigm, in order to ensure that the subjects are actually thinking about what they perceive on their intact hand, the method used to present the target should not provide any indication of the absolute target value. If an absolute value is provided, then the subject may end up matching the stimulation frequencies to the visual target instead of what is perceived. In one embodiment, the software module includes a visual target display system in which the actual value is relayed back to the subject as a color code that is computed relative to the assigned target level. That is, the subject is shown an object (e.g., a rectangular box) and is instructed to adjust the hand opening or pinch force on his or her intact hand such that the object color changes to a predetermined color (e.g., white). If the actual value is below the target then the color is a different predetermined color (e.g., shade of blue, such that darker shades indicate further distance from the target level), and if the actual value is above the target then it is yet another different color (e.g., a shade of red, such that darker shades indicate further distance from the target level). FIG. 15 shows an example of this in which the object is a rectangular box, the color for the target value is white, the color for an actual value below the target is a shade of blue (such that darker shades indicate further distance from the target level), and the color for an actual value below the target is a shade of red (such that darker shades indicate further distance from the target level). The software can include an option of changing the color scheme as desired.

Additionally, referring again to FIG. 8, in one embodiment the software module reads in the value from the foot pedal and maps it to the appropriate stimulation frequency. The mapped frequency value is passed on to the implanted stimulator through the external processor. During the course of this routine, a subject is presented with different target values and for each target value, the subject adjusts the stimulation using the foot pedal to match the perceptions on both arms. The software module logs in the adjusted stimulation values. At the end of the routine, the software module may include an option to plot the recorded pulse frequency values as a function of perception levels. The software module may automatically show the plot at the end of the routine, or it may have an option where the experimenter can manually choose to view and save the plot.

In one embodiment, the software module includes options to indicate the duration of the trial to the experimenter. The software can also include options to configure the sensors (such as those in FIGS. 9A and 9B) attached to the system.

In one embodiment, a system is designed to operate in “online mode” where the stimulation commands are provided by the custom software running on a computing device. For example, the online mode can be used mainly for the fitting process when the subject is in the lab. In an alternative embodiment, a system operates in “standalone mode” where the stimulation commands are computed by the processor in the NSS based on inputs from the sensors in the IPH. For example, standalone mode may be used by the subject during normal day-to-day tasks.

Embodiments of the subject invention are applicable for a hand prosthesis system with a prosthetic hand instrumented with any type of sensor known in the art, including temperature sensors, touch sensors, vibration sensors, pressure sensors, and/or other biologically relevant sensors known in the art. Similarly, the systems and methods described herein can be used with implanted stimulators fitted with any type of extraneural or intraneural electrodes.

Embodiments of the subject invention provide fitting methods and fitting systems for a prosthesis (e.g., a hand prosthesis) to the sensory fibers of the upper extremity using electrical stimulation. Any mode of stimulation, such as optical, magnetic, chemical, or other that can provide perception through activation of neural sensory fibers could be utilized. A similar procedure can be utilized for fitting a prosthesis for any portion of the amputated limb or part thereof. In addition, the fitting methods or systems could be used for activation of any central tissue, such as the brain or spinal cord, that is responsive to sensory stimulation.

Embodiments of the subject invention provide fitting methods, systems, and devices for fitting a limb prosthesis. The limb prosthesis can be for, e.g., an arm, a leg, a foot, or a hand. The prosthesis can be linked to the nervous system of a subject wearing the prosthesis.

In some embodiments, the bilateral tasks (perceptual modality paradigm) listed for sensory mapping can further be extended to fit a prosthesis which is controlled through recorded neural activity. Specifically, in an embodiment of the bilateral ‘mirrored’ action paradigm, a unilateral amputee with electrodes implanted on the amputated side and external sensors on the intact side could be asked to imagine doing the same set of tasks on both arms. The recorded neural data can be correlated with the information from the sensor to determine the mapping between neural activity and degree of motor intent. FIG. 16 is a block diagram showing an experimental setup according to an embodiment of the bilateral ‘mirrored’ action paradigm. A similar paradigm can be used in bilateral amputees or people with multiple limbs amputated, in which recorded neural data can be correlated to motor intent recorded from sensors that monitor signals from one or more location on the body (e.g., foot, head, tongue, and/or eye). Alternatively, the recorded neural activity can be displayed to the amputee and the mapping can be obtained using real-time biofeedback of the neural activity patterns.

In one embodiment, a fitting system for a motorized prosthesis can include a prosthesis having motors; and a neural recording system (NRS) comprising a recorder and electrodes in operable communication with the recorder. The motorized prosthesis can be in operable communication with the NRS.

In one embodiment, a fitting method for neural control of a motorized prosthesis with or without sensors can include the steps of: a) recording neural signals from one or more electrodes in the nervous tissue by a neural recording system (NRS) in operable communication with the electrodes; b) determining the neural signal level related to the threshold motor intent for the imagined motor task; c) determining the neural signal level related to the maximum motor intent for the imagined motor task; d) determining the mapping function that relates neural signal level to levels of motor intent between threshold and maximum; and e) delivering a signal to one or more motors of the prosthesis in accordance with mapping function developed using b), c), and d).

The methods and processes described herein can be embodied as code and/or data. The software code and data described herein can be stored on one or more computer readable media, which may include be any device or medium that can store code and/or data for use by a computer system. When a computer system reads and executes the code and/or data stored on a computer-readable medium, the computer system performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); or other media now known or later developed that is capable of storing computer-readable information/data. Computer-readable media should not be construed or interpreted to include any propagating signals.

All patents, patent applications, provisional applications, and publications referred to or cited herein, including those listed in the “References” section, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### REFERENCES

Atkins D J, Heard D C Y, Donovan W H (1996) Epidemiologic overview of individuals with upper-limb loss and their reported research priorities. J Prosthetics & Orthodics 8:2-11.
Bhaskaranand K, Bhat A K, Acharya K N (2003) Prosthetic rehabilitation in traumatic upper limb amputees (an Indian perspective). Arch Orthop Trauma Surg 123:363-366.
Clippinger F W, Avery R, Titus B R (1974) A sensory feedback system for an upper-limb amputation prosthesis. Bull Prosthet Res: 247-258.
Dhillon G S, Horch K W (2005) Direct neural sensory feedback and control of a prosthetic arm. IEEE Trans Neural Syst Rehabil Eng 13:468-472.
Dhillon G S, Lawrence S M, Hutchinson D T, Horch K W (2004) Residual function in peripheral nervestumps of amputees: implications for neural control of artificial limbs. J Hand Surg [Am] 29:605-615; discussion 616-608.
Dhillon G S, Kruger T B, Sandhu J S, Horch K W (2005) Effects of short-term training on sensory and motor function in severed nerves of long-term human amputees. J Neurophysiol 93:2625-2633.
Esquinazi A, Meier R (1996) Rehabilitation in limb deficiency. 4. limb amputation. Arch Phys Med Rehabil 77:S18-24.
Kerkovich D M (2004) Guest Editorial: A report on the amputee healthcare and prosthetics workshop sponsored by Walter Reed Army Medical Center and the Department of Veterans Affairs (VA). J Rehabil Res Dev 41:xiii-xvi.
Kuiken T A, Dumanian G A, Lipschutz R D, Miller L A, Stubblefield K A (2004) The use of targeted muscle reinnervation for improved myoelectric prosthesis control in a bilateral shoulder disarticulation amputee. Prosthet Orthot Int 28:245-253.
Goodall E, Lefurge T, Horch K (1991) Information contained in sensory nerve recordings made with intrafascicular electrodes. IEEE Trans Biomed Eng 38:846-850.
Lefurge T, Goodall E, Horch K, Stensaas L, Schoenberg A (1991) Chronically implanted intrafascicular recording electrodes. Ann Biomed Eng 19:197-207.
Childress D S (1973) Powered limb prostheses: Their clinical significance. IEEE Trans Biomed Eng BME-20:200-207.
Mann R W (1973) Prostheses control and feedback via non-invasive skin and invasive peripheral nerve techniques. In: Neural Organization and its Relevance to Prosthetics (Fields W S, ed), pp 177-195. New York: Intercontinental Medical Books.
Hoffer J A (2001) How to Use Nerve Cuffs to Stimulate, Record or Modulate Neural Activity. In: Neural Prostheses for Restoration of Sensory and Motor Function (Chapin J K, ed), pp 139-178. New York: CRC Press.
Carruthers M T, Pottinger J M (1969) Research into the possibility of a substitute for proprioception. Proc Instn Mech Engrs 183:98-102.
Sahin M, Durand D M (1998) Improved nerve cuff electrode recordings with subthreshold anodic currents. IEEE Trans Biomed Eng 45:1044-1050.
Yoshida K, Riso R (2004) Peripheral nerve recording electrodes and techniques. In: Neuroprosthetics: Theory and Practice (Horch K W, Dhillon G S, eds): World Scientific.
Zheng X, Zhang J, Chen T, Chen Z (2003) Longitudinally implanted intrafascicular electrodes for stimulating and recording fascicular physioelectrical signals in the sciatic nerve of rabbits. Microsurgery 23:268-273.
Mensinger A F, Anderson D J, Buchko C J, Johnson M A, Martin D C, Tresco P A, Silver R B, Highstein S M (2000) Chronic recording of regenerating VIIIth nerve axons with a sieve electrode. J Neurophysiol 83:611-615.
Kovacs G T, Storment C W, Rosen J M (1992) Regeneration microelectrode array for peripheral nerve recording and stimulation. IEEE Trans Biomed Eng 39:893-902.
Edell D J (1986) A peripheral nerve information transducer for amputees: long-term multichannel recordings from rabbit peripheral nerves. IEEE Trans Biomed Eng 33:203-214.
Branner A, Normann R A (2000) A multielectrode array for intrafascicular recording and stimulation in sciatic nerve of cats. Brain Res Bull 51:293-306.
Malagodi M S, Horch K W, Schoenberg A A (1989) An intrafascicular electrode for recording of action potentials in peripheral nerves. Ann Biomed Eng 17:397-410.
Malmstrom J A, McNaughton T G, Horch K W (1998) Recording properties and biocompatibility of chronically implanted polymer-based intrafascicular electrodes. Ann Biomed Eng 26:1055-1064.
Branner A, Stein R B, Fernandez E, Aoyagi Y, Normann R A (2004) Long-term stimulation and recording with a penetrating microelectrode array in cat sciatic nerve. IEEE Trans Biomed Eng 51:146-157.
U.S. Pat. Nos. 6,609,032, 7,302,296, and 7,908,011

1. A fitting system for a prosthesis, comprising:
an instrumented prosthesis (IP) having sensors configured to sense modalities of the IP; and
a neural stimulation system (NSS) comprising a stimulator and electrodes in operable communication with the stimulator,
wherein the IP is in operable communication with the NSS.

2. The fitting system according to claim 1, wherein the NSS comprises a processor, wherein the stimulator is an implantable stimulator, and wherein the electrodes are longitudinal intrafascicular electrodes (LIFEs).

3. The fitting system according to claim 1, further comprising a software component comprising a software module stored on a computer readable medium, wherein the NSS is in operable communication with the software component.

4. The fitting system according to claim 3, wherein the software component comprises a user interface (UI), and wherein the software module comprises of at least one of the following:
a startup module; an impedance module; a calibration module; a fitting module; a configuration module; and a safety module.

5. The fitting system according to claim 4,
wherein the startup module allows a user of the software component, via the UI, to perform at least one of the following operations: create a new subject database; edit existing subject information; choose a subject to start an experimental session; load previous session information; and archive current session information,
wherein the impedance module allows the user of the software component, via the UI, to perform at least one of the following operations: measure impedance of at least one of the electrodes of the NSS; display the impedance value of at least one of the electrodes of the NSS; and display the status of at least one of the electrodes of the NSS,
wherein the calibration module comprises at least one of the following portions: a subject entry section for the subject to enter data; a parameter section for setting the value of at least one parameter; a test mode section for entering information related to testing; a pulse timing section for entering and/or displaying information related to the electrical impulses of the stimulator; an electrode information section for entering and/or displaying information related to at least one electrode of the NSS; and a display section for displaying a graphical representation of stimulation values presented to the subject during a calibration process,
wherein the fitting module allows the user of the software component, via the UI, to perform at least one of the following operations: choose at least one electrode linked to a particular sensory modality; and choose a text file with a list of stimulation parameter values to be tested,
wherein the configuration module allows the user of the software component, via the UI, to configure the NSS with at least one of the following: electrode calibration; mapping parameters; and timing parameters, and
wherein the safety module allows the user of the software component, via the UI, to configure checks that ensure that stimulation parameters passed onto the stimulator during use are within predetermined safety limits.

6. The fitting system according to claim 3, wherein the software component comprises a UI, and wherein the UI comprises at least one of the following: a visual representation of electrode placement in a nerve; means of selecting one or more electrodes for calibration, wherein selected electrodes for stimulation provide a perception of sensation of a particular modality; and means of mapping information from one type of sensor to stimulation values on one or more electrodes linked to a corresponding sensory modality.

7. The fitting system according to claim 6,
wherein the software module comprises a means of selecting one or more electrodes to check electrode integrity by estimating electrode impedance,
wherein a second section of the UI comprises at least one of the following:
controls to select a specific calibration routine for a particular electrode of the NSS and adjust routine configuration parameters;
a display of stimulation parameters; and
a means by which a user can graphically mark a location and modality of sensation, and
wherein a third section of the UI comprises at least one of the following:
options to generate a list of target values for a mapping paradigm;
an option of retrieving a previously generated target list;
options to provide the user and a subject on whom the sensors are connected with a visual or other sensory information of data obtained from the sensors; and
options to configure the sensors.

8. The fitting system according to claim 3, wherein the software component comprises a UI, and wherein the UI comprises sections configured to allow a user to perform at least one of the following operations: measure electrode impedances; determine electrode integrity; calibrate individual electrodes or a group of electrodes; link electrodes to a particular sensory modality; map perception levels to stimulation parameters on one or more electrodes; and configure the IP.

9. The fitting system according to claim 1, wherein the NSS is configured to receive sensor values from the sensors of the IP and to map the received sensor values to stimulation parameter values for the electrodes of the NSS.

10. The fitting system according to claim 1, wherein the electrodes are positioned on a subject adjacent to sensory fibers corresponding to the sensors of the IP.

11. The fitting system according to claim 1, wherein the processor of the NSS is in operable communication with the stimulator using a radio frequency (RF) link or other wireless communication link.

12. The fitting system according to claim 1, wherein the processor of the NSS is in operable communication with the stimulator using a wired communication link.

13. A fitting method for an instrumented prosthesis (IP), the method comprising the steps of:
receiving, by a processor of a neural stimulation system (NSS), at least one sensor value from sensors of the IP;
mapping the at least one sensor value to at least one stimulation parameter value on one or more electrodes of the NSS;
providing the stimulation parameter value to a stimulator of the NSS in operable communication with the electrodes; and
delivering, by the one or more electrodes, an electrical impulse to a peripheral nerve of a subject having the IP, in accordance with the at least one stimulation parameter value.

14. The fitting method according to claim 13, wherein the processor of the NSS is in operable communication with the stimulator using a radio frequency (RF) link or other wireless communication link.

15. The fitting method according to claim 13, further comprising a calibration step to determine a limit of the at least one stimulation parameter.

16. The fitting method according to claim 15, wherein the calibration step comprises determining at least one of the following: amplitude threshold; amplitude saturation; stimulation amplitude; frequency threshold; frequency high; frequency saturation; n-let pulse parameters; specification of jitter on interspike intervals; and specification of interchannel spike timing.

17. The fitting method according to claim 16, wherein the calibration step comprises performing at least one of the following operations:
a) determining the amplitude threshold using a staircase procedure or a method of limits by presenting the subject with a series of pulses;
b) determining the amplitude saturation using a staircase procedure or a method of limits by presenting the subject with a series of pulses;
c) determining the stimulation amplitude by averaging an amplitude threshold value and an amplitude saturation value or by taking a percentage of a total range of amplitude values;
d) determining the frequency threshold using a staircase procedure or a method of limits by presenting the subject with a series of pulses;
e) determining the frequency high by presenting the subject with a series of pulses ; and
f) determining the frequency saturation using a just noticeable difference procedure.

18. The fitting method according to claim 17, wherein each of operations a), b), d), e), and f) further comprises the subject providing input to a software component stored on a computer readable medium and in operable communication with the NSS,
wherein the subject provides the input through a user interface (UI) of the software component, and
wherein the input provided by the subject is used to determine the amplitude threshold, the amplitude saturation, the frequency threshold, the frequency high, and/or the frequency saturation.

19. The fitting method according to claim 13, wherein mapping the at least one sensor value to at least one stimulation parameter value comprises having the subject match perception of a given modality that is elicited by electrical or other modes of stimulation on one site of the subject's body with the same modality on another site of the subject's body.

20. The fitting method according to claim 13, wherein mapping the at least one sensor value to at least one stimulation parameter value comprises:
having the subject position the subject's intact side to match a target posture; and
adjusting stimulation on the other side of the body to match perception of the intact side.
